(19) United States
(12) Patent Application Publication
(10) Pub. No.: US 2023/0018524 A1
(43) Pub. Date: Jan. 19, 2023

(54) MULTIMODAL CONVERSATIONAL PLATFORM FOR REMOTE PATIENT DIAGNOSIS AND MONITORING

(71) Applicant: MODALITY.AI, INC., San Francisco, CA (US)

(72) Inventors: Vikram Ramanarayanan, Berkeley, CA (US); Oliver Roesler, Weyhe (DE); Michael Neumann, Waiblingen (DE); David Pautler, San Francisco, CA (US); Doug Habberstad, Hilton Head, SC (US); Andrew Cornish, Gore (NZ); Hardik Kothare, Burlingame, CA (US); Vignesh Murali, Cambridge, MA (US); Jackson Liscombe, New Marlborough, MA (US); Dirk Schnelle-Walka, Rhineland-Palatinate (DE); Patrick Lange, San Francisco, CA (US); David Suendermann-Oeft, San Francisco, CA (US)

(21) Appl. No.: 17/508,693

(22) Filed: Oct. 22, 2021

Related U.S. Application Data
(60) Provisional application No. 63/223,424, filed on Jul. 19, 2021.

(51) Int. Cl. A61B 5/00; G16H 50/30; G10L 25/66; G06F 40/18

(52) U.S. Cl. CPC A61B 5/4803; G16H 50/30; G10L 25/66; G06F 40/18

(57) ABSTRACT

A virtual agent instructs a responding person to perform specific verbal exercises. Audio and image inputs from the responding person's performance of the exercises are used to identify speech, video, cognitive, and/or respiratory biomarkers, which are then used to evaluate speech motor function and/or neurological health. Contemplated exercises include test aspects of oral motor proficiency, sustained phonation, diadochokinesis, reading speech, spontaneous speech, spirometry, picture description, and emotion elicitation. Metrics from evaluation of the responding person's performance are advantageously produced automatically, and are presented in spreadsheet format.

This application claims priority to provisional patent application Ser. No. 63/223,424, filed on Jul. 13, 2021. The provisional and all other referenced extrinsic materials are incorporated herein by reference in their entirety. Where a definition or use of a term in a reference that is incorporated by reference is inconsistent or contrary to the definition of that term provided herein, the definition of that term provided herein is deemed to be controlling.

### FIELD OF THE INVENTION

The field of the invention is healthcare informatics, especially analysis of psychological or other medical conditions.

### BACKGROUND

The following description includes information that may be useful in understanding the present invention. It is not an admission that any of the information provided herein is prior art or relevant to the presently claimed invention, or that any publication specifically or implicitly referenced is prior art.

Diagnosis, detection, and monitoring of medically-related conditions remain a critical need. The problems are often exacerbated by: (i) lack of access to neurologists or psychiatrists; (ii) lack of awareness of a given condition and the need to see a specialist; (iii) lack of an effective standardized diagnostic or endpoint for many of these health conditions; (iv) substantial transportation and cost involved in conventional or traditional solutions; and in some cases, (v) shortage of medical specialists in these fields.

There have been many efforts to address these problems, including use of telemedicine, in which a practitioner interacts with a patient or patients utilizing telecommunications. Telemedicine does not, however, resolve problems associated with insufficient numbers of trained practitioners, or available time of existing practitioners. Psychological conditions, in particular, can often require lengthy times spent with responding patients. Current systems for telemedicine also fail to address inadequacies in electronic communications, especially in rural areas where adequate line speed and reliability are lacking.

As used herein, the term “patient” means any person with which a human or virtual practitioner is communicating with respect to a psychological or other condition, or potential such conditions, even if the person has not been diagnosed, and is not under the care of any practitioner. A patient is also from time to time herein referred to as a “responding person”.

As used herein, the term “practitioner” broadly refers to any person whose vocation involves diagnosing, treating, or otherwise assisting in assessing or remediating psychological and/or other medical issues. In this usage, practitioners are not limited to medical doctors or nurses, or other degreed providers. Still further, as used herein, “medical conditions” should be interpreted as including psychological conditions, regardless of whether such conditions have any underlying physical etiology.

As used herein, the terms “assessment”, “assessing”, and related terms means weighing information from which at least a tentative conclusion can be drawn. The at least tentative conclusion need not rise to the level of a formal diagnosis.

As used herein, the term “virtual agent” broadly refers to a computer or other non-human functionality configured to operate as a practitioner in assessing or remediating psychological and/or other medical issues. Virtual agents having functionalities augmented by one or more humans are still considered herein to be virtual agents.

Pending U.S. patent application Ser. No. 17/471,929, “Use Of Virtual Agent To Assess Psychological And Medical Conditions” describes apparatus, systems, and methods in which a virtual agent converses with a responding person to assess one or more psychological or other medical conditions of the responding person. The virtual agent uses both semantic and affect content from the responding person to branch the conversation, and also to interact with a data store to provide an assessment of the medical or psychological condition.

The '929 application taught deriving semantic and/or affect content from evaluating a patient's response during a conversational question session. Responses evaluated included facial expressions, eye movements, extent of eye contact, posture, hand gestures, and audible speech. Evaluated speech characteristics included voice pitch, voice speed, voice loudness, and a non-verbal utterance.

Research and development has continued, and the inventors herein have discovered that structured conversation exercises can be automatically utilized to provide objective, scalable, and repeatable assistance in assessing psychological and medical conditions

### SUMMARY OF THE INVENTION

The inventive subject matter provides a multimodal conversational platform for remote patient diagnosis and monitoring. The platform engages patients in an interactive dialog session and automatically computes metrics relevant to speech acoustics and articulation, oro-motor and oro-facial movement, cognitive function and respiratory function. The dialog session includes a selection of exercises that have been widely used in both speech language pathology research as well as clinical practice—an oral motor exam, sustained phonation, diadochokinesis, read speech, spontaneous speech, spirometry, picture description, emotion elicitation and other cognitive tasks. Finally, the system automatically computes speech, video, cognitive and respiratory biomarkers that have been shown to be useful in capturing various aspects of speech motor function and neurological health and visualizes them in a responding person-friendly dashboard.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

FIG. 1A is a schematic of an assessment session in which of a virtual agent instructs a patient to repeat a simple phrase until he/she runs out of breath.

FIG. 1B is a schematic of an assessment session in which of a virtual agent instructs a patient to repeat a longer phrase.

FIG. 1C is a schematic of an assessment session in which of a virtual agent instructs a patient to read a written paragraph.

FIG. 2 is a listing of contemplated exercises.

FIG. 3 is a portion of an exemplary dashboard showing a tabular display of metrics derived from a patient's performance of instructed exercises.

FIG. 4 is a flowchart of a practitioner and/or a virtual agent virtual agent instructing a patient to execute verbal exercises.

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously.

As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise.

All methods described herein can be performed in any suitable order unless otherwise indicated herein or otherwise clearly contradicted by context. The use of any and all examples, or exemplary language (e.g. “such as”) provided with respect to certain embodiments herein is intended merely to better illuminate the invention and does not pose a limitation on the scope of the invention otherwise claimed. No language in the specification should be construed as indicating any non-claimed element essential to the practice of the invention. Unless a contrary meaning is explicitly stated, all ranges are inclusive of their endpoints, and open-ended ranges are to be interpreted as bounded on the open end by commercially feasible embodiments.

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

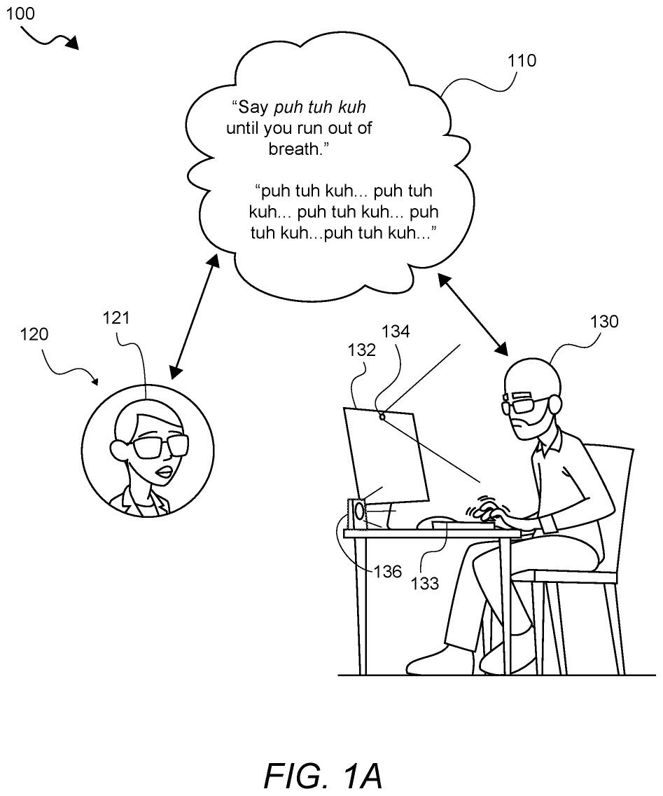

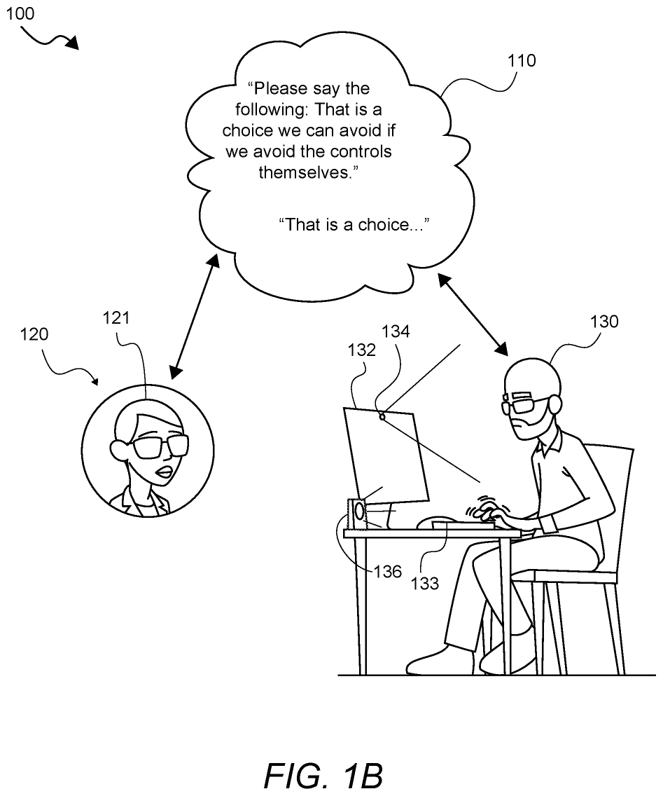

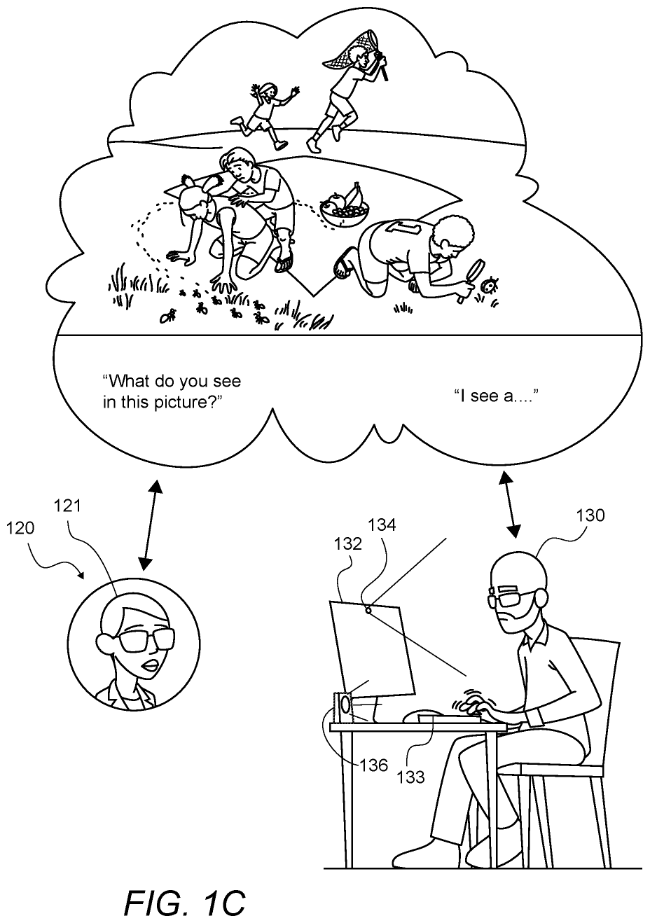

FIGS. 1A-1C are schematic views 100 of a virtual agent 120 conducting an assessment session with a responding person 130 through electronic means 110, over cloud 120. In each instance, the virtual agent 120 instructs a responding person 130 to perform specific verbal exercises. Audio and image inputs from the responding person's performance of the exercises are used to identify speech, video, cognitive, and/or respiratory biomarkers, which are then used to evaluate speech motor function and/or neurological health. Contemplated exercises include test aspects of oral motor proficiency, sustained phonation, diadochokinesis, reading speech, spontaneous speech, spirometry,

FIGS. 1A-1C are different in that they depict instructions and responses with respect to different types of exercises. In FIG. 1A, the exercise involves the responding person 130 repeating a short phrase over and over until he/she runs out of breath. In FIG. 1B, the exercise involves the responding person 130 reading a paragraph. In FIG. 1C, the exercise involves the responding person 130 providing his/her interpretation of a visual scene.

Although virtual agent 120 can be presented simplistically to the responding person 130 as a disembodied voice, or perhaps a still image or cartoon (not shown), virtual agent 120 is preferably presented in a more realistic approximation of a live person. In FIGS. 1A-1C, virtual agent 120 is depicted as a CGI avatar 121, sitting in front of a CGI computer 122 with an optional CGI keyboard 123, a CGI combination camera/microphone 124, and a CGI speaker 126. In FIGS. 1A-1C the avatar 121 is depicted as a middle aged woman, however the avatar 121 could alternatively be depicted as a human of any other age and gender, or even an animal or other non-human character.

Virtual agent 120 should be interpreted as including one or more processors storing and executing instructions on one or more computer readable, non-transitory storage devices. Contemplated computing and storage devices include one or more computers operating as a web server, database server, or other type of computer server, and related storage devices, and can be physically local to one another, or more likely are distributed in different cities and even different countries. Although virtual agent 120 is depicted as interacting with a single responding person 130, virtual agent 120 should be interpreted as being configured in a cloud or other computing environment that allows virtual agent 120 to concurrently assess multiple responding persons.

Cloud 110 should be viewed generically as any suitable communications network, over which are traveling communications between the virtual agent 120 and the responding person 130.

In FIGS. 1A-1C, responding person 130 is a physical person, and is using a communication device to communicate with the virtual agent 120. The communication device is represented as a desktop computer 132 with a keyboard 133, a transmitting camera/microphone 134, and a speaker 136. However, these components should be viewed generically to include any suitable device or devices fulfilling their usual functions, including for example a laptop, an iPad™ or other tablet, and even a cell phone.

Although responding person 130 is depicted as sitting at a desk, it is contemplated that responding person 130 could be interacting in any suitable posture, including for example, walking about, sitting on a couch, or lying in bed. However, it is important that responding person 130 is situated with respect to the camera and microphone such that the virtual agent can obtain sufficient information from the responding person's lip and other facial movements, and speech characteristics.

Although responding person 130 is shown as an older man, FIGS. 1A-1C) should be viewed broadly enough to include all realistic ages and genders for responding person.

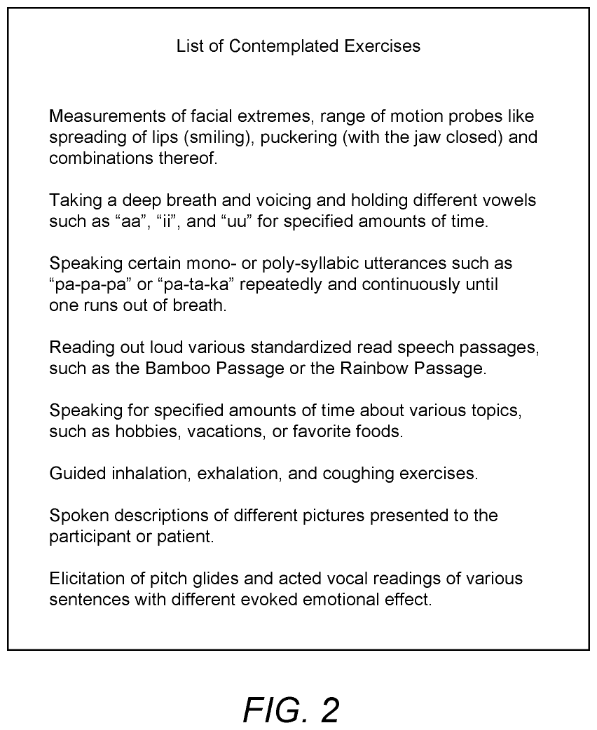

FIG. 2 is a listing of contemplated exercises.

Contemplated oral motor exercises include, but are not limited to, measurements of facial extremes, range of motion probes like spreading of lips (smiling), puckering (with the jaw closed) and combinations thereof.

Contemplated sustained phonation exercises include, but are not limited to, taking a deep breath and voicing and holding different vowels such as “aa”, “ii” and “uu” for specified amounts of time.

Contemplated diadochokinesis exercises include, but are not limited to, speaking certain mono- or poly-syllabic utterances such as “pa-pa-pa” or “pa-to-ka” repeatedly and continuously until one runs out of breath.

Contemplated read speech exercises include, but are not limited to, reading out loud various standardized read speech passages, such as the Bamboo Passage or the Rainbow Passage.

Contemplated spontaneous speech exercises include, but are not limited to, speaking for specified amounts of time about various topics, such as hobbies, vacations or favorite foods.

Contemplated spirometry exercises include, but are not limited to, guided inhalation, exhalation and coughing exercises.

Contemplated picture description exercises include, but are not limited to, spoken descriptions of different pictures presented to the participant or patient.

Contemplated emotion elicitation exercises include, but are not limited to, elicitation of pitch glides and acted vocal readings of various sentences with different evoked emotional affect.

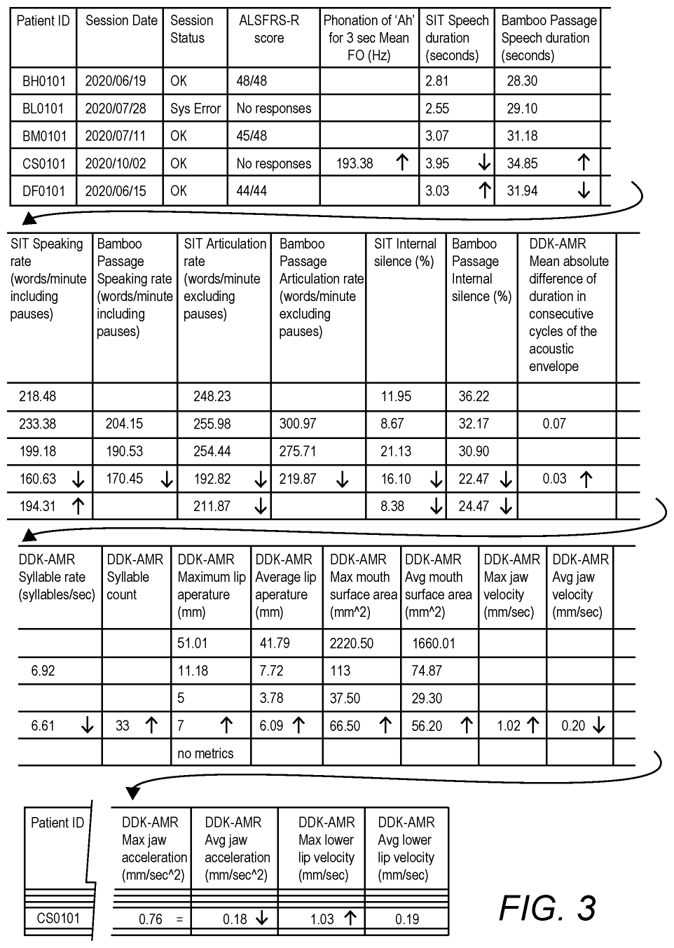

FIG. 3 is a portion of an exemplary dashboard showing a tabular display of metrics derived from a responding person's performance of the instructed exercises. In this example, the column headings identify speech and facial biomarkers that are appropriate and informative to extract for a given project, and the rows depict responding party identifications, and metrics automatically determined from the performances of the responding persons. It should be appreciated that the columns depicted in FIG. 3 are merely for illustrative purposes. In practice, dashboards would likely 100 or more columns.

It should also be appreciated that practice of the concepts disclosed herein are especially valuable when communication with responding persons is executed entirely or almost entirely automatically, and assessment of the various performances to produce metrics as in FIG. 3 is also executed entirely or almost entirely automatically. Automatic assessment of the various performances to produce metrics can be accomplished in any suitable manner, and especially through utilization of the following data stores and analytic programs:
I. Yunusova et al (2011). A Protocol for Comprehensive Assessment of Bulbar Dysfunction in Amyotrophic Lateral Sclerosis (ALS). (https://www.ncbi.nlm.nih.gov/pmc/articles/PMC3197394/)
II. Mundt et al (2007) (https://www.ncbi.nlm.nih.gov/pmc/articles/PMC3022333/)
III. Vasquez-Correa et al (2017) https://www5.informatik.uni-erlangen.de/Forschung/Publikationen/2018/Vasquez-Correa18-TAA.pdf

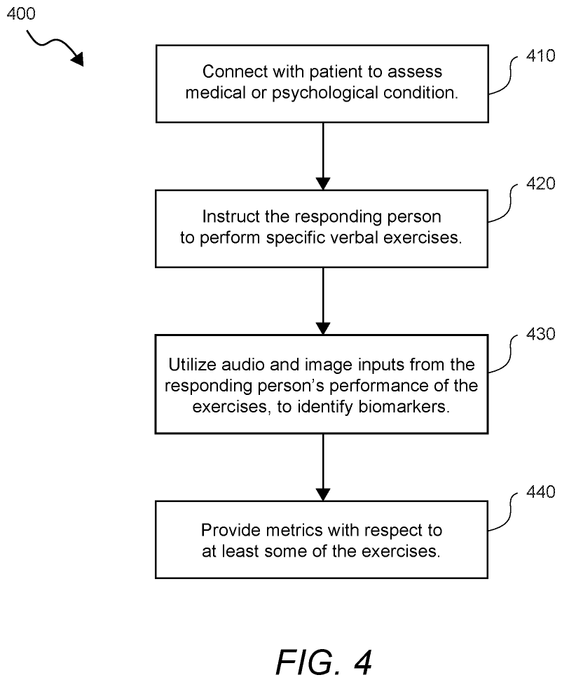

FIG. 4 is a flowchart 400 of a practitioner and/or a virtual agent virtual agent instructing a patient/responding person to execute verbal exercise having the following steps: Step 410—Connect with patient to assess medical or psychological condition; Step 420—Instruct the responding person to perform specific verbal exercises; Step 430—Utilize audio and image inputs from the responding person's performance of the exercises, to identify biomarkers; and Step 440—Provide metrics with respect to at least some of the exercises.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. Where the specification refers to at least one of something selected from the group consisting of A, B, C . . . and N, the text should be interpreted as requiring only one element from the group, not A plus N, or B plus N, etc.

What is claimed is:

1. A method of assessing a medical or psychological condition of a responding person,
comprising configuring a processor to execute instructions that operate a virtual agent configured to:
instruct the responding person to perform specific verbal exercises;
utilize audio and image inputs from the responding person's performance of the exercises, to identify at least one of speech, video, cognitive, and respiratory biomarkers with respect to at least one of speech motor function and neurological health; and
providing metrics corresponding to the responding person's performance with respect to at least some of the exercises.

2. The method of claim 1, wherein at least one of the exercises is selected to test aspects of oral motor proficiency.

3. The method of claim 1, wherein at least one of the exercises is selected to test aspects of sustained phonation.

4. The method of claim 1, wherein at least one of the exercises is selected to test aspects of diadochokinesis

5. The method of claim 1, wherein at least one of the exercises is selected to test aspects of reading speech.

6. The method of claim 1, wherein at least one of the exercises is selected to test aspects of spontaneous speech.

7. The method of claim 1, wherein at least one of the exercises is selected to test aspects of spirometry.

8. The method of claim 1, wherein at least one of the exercises is selected to test aspects of picture description.

9. The method of claim 1, wherein at least one of the exercises is selected to test aspects of emotion elicitation

10. The method of claim 1, further comprising rendering the metrics in a spreadsheet format.

11. The method of claim 1, wherein the utilizing the audio and image inputs is completely automatic.
